(19) United States
(12) Patent Application Publication
(10) Pub. No.: US 2003/0236480 A1
(43) Pub. Date: Dec. 25, 2003

(54) Preformed nasal septum skin barrier device

(76) Inventor: Robert M. Landis, Mountainside, NJ (US)

Correspondence Address: DUANE MORRIS LLP, 100 COLLEGE ROAD WEST, SUITE 100, PRINCETON, NJ, 08540-6604, US

(21) Appl. No.: 10/178,452

(22) Filed: Jun. 24, 2002

(51) Int. Cl. A61F 15/00

(52) U.S. Cl. 602/54

(57) ABSTRACT

In general, the invention relates to a preformed nasal septum skin barrier device having a mounting member and a stem portion which extends outwardly from the mounting member. The device also has an adhesive means, associated with at least the mounting member, for applying the device to an area adjacent an upper lip of an individual. When so applied the stem portion covers at least a portion of a nasal septum area of the individual and prevents nasal cannulae from contacting and causing an injury to the nasal septum area. More particularly, the stem portion may extend substantially perpendicularly from the mounting member. Additionally, the adhesive means for applying the device can be a skin compatible adhesive, covered by a removable protective covering, which is disposed on the mounting member and/or the stem portion.

### FIELD OF THE INVENTION

The present invention relates to a skin barrier device and more specifically, to a preformed nasal septum skin barrier device for use with a nasal cannula to prevent skin abrasions or injury to the nasal septum area.

### BACKGROUND OF THE INVENTION

Nasal cannulae are well known in the art and are conventionally used to provide continuous positive airway pressure (CPAP) and/or airflow assistance to individuals. Such nasal cannulae assemblies typically comprise a hollow, tubular cannula body, having a pair of prongs, or outlets, extending from the cannula body. The prongs are placed within an individual's nasal passages to deliver air, oxygen or other gases as a respiratory supplement.

Such nasal cannula asemblies are often used over a long period of time. It is frequently necessary to wear the cannula on a 24-hour a day basis. Such continuous and prolonged usage commonly results in abrasion, irritation, sores, discomfort and other injuries in areas where the cannula contacts the skin. In particular, the rubbing of the cannula against the nasal septum area often causes trauma to that area.

Infants, and more particularly prematurely born infants, are especially susceptible to such problems. Due to their small size, the prongs of the cannula cover a larger cross-section of their nasal orifices, or nares. Additionally, infants under six months of age typically breathe entirely through their noses. Thus, the prongs and associated parts of the cannula body tend to cause pressure contact injuries to the nasal passages and septum areas after prolonged usage. In some cases, the resulting deterioration is so severe that it requires repair through plastic surgery.

Various prior art systems have attempted to address this difficulty. Typically, such systems involve variations on cannula or nosepiece designs. As an example, a technique disclosed in U.S. Pat. No. 3,802,431 provides a nasal cannula assembly having a flexible, curved lip plate extending from the cannula body for positioning the cannula on the patient's upper lip, which also serves to reduce skin contact with the cannula. U.S. Pat. No. 3,643,660 shows nasal cannulae having hollow tubular bodies with a flat or plane surface to provide a smooth surface for contacting the patient's upper lip.

However, such prior art systems require relatively complicated cannula or nosepiece designs. Such designs may not always be convenient or available for use, or they may be more expensive than conventional designs. Additionally, those techniques may still allow some degree of rubbing up of a cannula assembly member against the nasal septum area, resulting in injury to that area. Skin abrasions are especially likely for premature infants, who due to their softer skin and smaller size in relation to such devices are especially prone to experience such injury. Additional prior art variations, although they attempt to reduce trauma to the wearer, do not at all address the problem of damage to the nasal septum area.

U.S. Pat. No. 4,534,342 to Paxa shows a nose bandage for retaining nasal packing or other nasal occlusion devices and for use as a nasal-facial protective covering in the care of external nasal-facial injuries. However, this bandage is not intended to have the purpose of avoiding skin abrasions and other trauma resulting from the use of nasal cannulae or similar medical devices.

What is needed, therefore is an improved device for preventing the nasal cannulae from rubbing against the nasal septum and causing trauma to that area, particularly for use with neonates, prematurely born infants and other individuals who are highly susceptible to such injuries.

### SUMMARY OF THE INVENTION

The present invention relates to a preformed nasal septum skin barrier device having a mounting member and a stem portion, which extends outwardly from the mounting member. The device has an adhesive means for applying the device to an area adjacent an upper lip of an individual. When so applied the stem portion covers at least a portion of a nasal septum area of the individual and prevents nasal cannulae from contacting and causing an injury to the nasal septum area.

More particularly, the stem portion may extend substantially perpendicularly from the mounting member. Additionally, the adhesive means for applying the device can include a skin compatible adhesive, covered by a removable protective covering, which is disposed on the mounting member and/or the stem portion.

### BRIEF DESCRIPTION OF THE FIGURES

For a fuller understanding of the invention, reference is made to the following description taken in connection with the accompanying drawings, in which:

FIG. 1 illustrates a front perspective view of an infant's face utilizing the invention;

FIG. 2 illustrates a plan view of the present invention; and

FIG. 3 illustrates a side view of a human face utilizing the invention, with a cross sectional view of an associated nasal cannula.

### DETAILED DESCRIPTION OF THE INVENTION

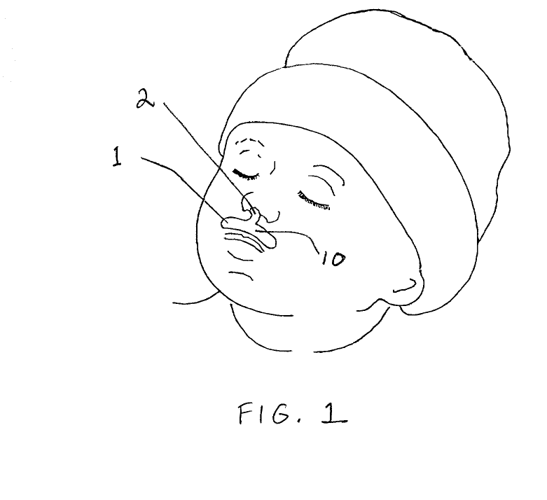

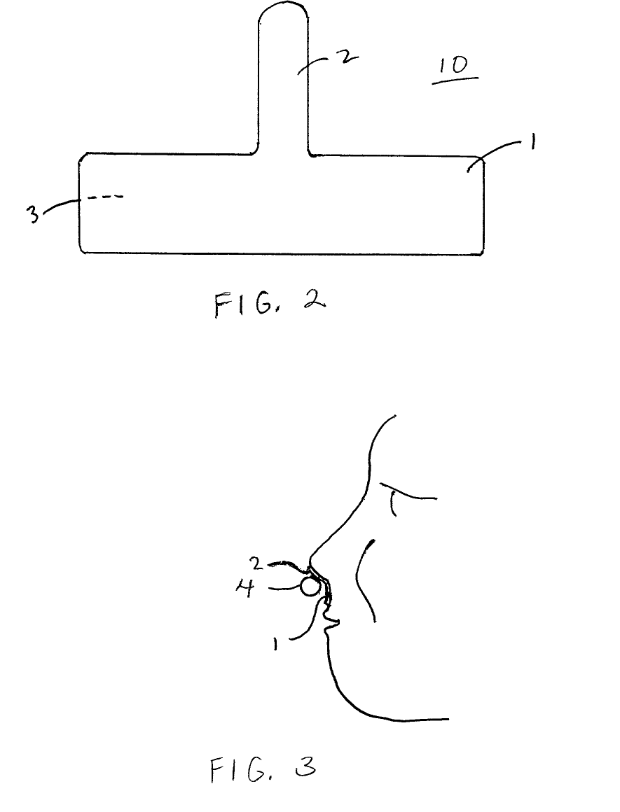

FIG. 1 shows the preformed nasal septum skin barrier device 10 of the present invention in use on the face of an infant. As illustrated in FIG. 2, the device 10 comprises a flexible mounting member 1 and an integral stem portion 2 extending substantially perpendicularly from the mounting member 1. In the illustrated embodiment, the mounting member 1 is an elongated member, and the stem portion 2 extends from a generally central area of the mounting member 1.

The device may be constructed of a thin, soft, foam like material and has an adhesive means for applying it to the upper lip and nasal septum areas of an individual. In the mode of the invention shown, such means comprises a pressure sensitive adhesive 3 coating one side of the device. The adhesive may be covered by a removable protective covering or backing. Such an adhesive is typically skin compatible and of a type that is releasable from the wearer's skin without causing damage to the skin. Such adhesives are well known and many examples exist in the prior art.

As indicated in FIG. 1, the adhesive means allows the application of the mounting member 1 to the upper lip area. The adhesive means also permits the application of the stem portion 2 to the nasal septum area in such a manner that the stem portion 2 covers at least a portion of the nasal septum area. In the aspect of the invention shown, the stem portion covers the nasal septum area so as to prevent a nasal cannula 4 from rubbing up against the septum and causing trauma to that area.

FIG. 3 illustrates a side view of a human face utilizing the invention, which also shows the positioning of the associated nasal cannula 4.

Thus, the subject invention covers both the nasal septum and upper lip and prevents skin abrasions and other injuries to those areas resulting from contact with a nasal cannula. The highly simple mode of the present device is significantly more effective and convenient to use than the complicated nasal cannulae designs described above. Medical personnel need only to remove the preformed device from its protective backing or covering and apply it to a wearer's face. Unlike the prior art cannulae designs, the device completely prevents contact of the cannula with the nasal septum area, thereby avoiding the injuries described above.

The design of the present invention is also much simpler than prior art nose bandages as mentioned above. The present invention offers a highly simple inverted T-shaped design. In addition, unlike prior art references, the present invention specifically addresses the problem of damage to the nasal septum area.

The mode of the invention shown also provides for improved cost effectiveness. The preformed skin barrier device offers a very low cost technique that is extremely convenient and easy to apply, as compared with prior art systems. There is no need for a technician or other medical personnel to expend more than a very minimal amount of time to prevent the trauma problems described above.

The foregoing description of the embodiments of this invention has been presented for purposes of illustration and description. It is not intended to be exhaustive or to limit the embodiments of the invention to the form disclosed, and, obviously, many modifications and variations are possible. For example, the device may be made of cloth, porous flexible material, woven adhesive material or other like material. Various adhesive means for applying the device, known in the art, may be used, and such means can be secured to the mounting member, the stem portion, or both.

It is also understood that the invention is meant to cover preformed nasal septum skin barrier devices having geometrical configurations other than the strict inverted T-shape described. Such modifications and variations that may be apparent to a person skilled in the art are intended to be included within the scope of this invention as defined by the accompanying claims.

What is claimed is:

1. A preformed nasal septum skin barrier device comprising:
a mounting member;
a stem portion, extending outwardly from the mounting member; and
an adhesive means associated with at least the mounting member, for applying the device to an area adjacent an upper lip of an individual in such a manner that the stem portion covers at least a portion of a nasal septum area of the individual to prevent a nasal cannula from contacting and causing an injury to the nasal septum area.

2. The preformed nasal septum skin barrier device of claim 1, where the adhesive means comprises a skin compatible adhesive.

3. The preformed nasal septum skin barrier device of claim 2, where the adhesive is covered by a removable protective covering.

4. The preformed nasal septum skin barrier device of claim 1, where the stem portion extends substantially perpendicularly from the mounting member.

5. The preformed nasal septum skin barrier device of claim 1, where the adhesive means is disposed on the mounting member.

6. The preformed nasal septum skin barrier device of claim 1, where the adhesive means is disposed on the stem portion.

7. A preformed nasal septum skin barrier device comprising:
a mounting member;
a stem portion, extending substantially perpendicularly from the mounting member; and
a skin-compatible adhesive, disposed on at least the mounting member and covered by a removable protective covering, for applying the device to an area adjacent an upper lip of an individual in such a manner that the stem portion covers at least a portion of a nasal septum area of the individual to prevent a nasal cannula from contacting and causing an injury to the nasal septum area.

8. A preformed nasal septum skin barrier device of claim 7, where the mounting member and the stem portion form an inverted T-shaped configuration.

9. A preformed nasal septum skin barrier device of claim 7, where the skin-compatible adhesive is disposed on the stem portion.
